SEQUENCE LISTING (43 sequences)

SEQ ID NO: 1 - DNA, 29 bp, Artificial Sequence
  Description of Artificial Sequence: Synthetic polynucleotide sequence
  3′-propylamine group may be attached
tttggtggtggtggttgtggtggtggtgg

SEQ ID NO: 2 - DNA, 29 bp, Artificial Sequence
  Description of Artificial Sequence: Synthetic polynucleotide sequence
tttggtggtggtggttttggtggtggtgg

SEQ ID NO: 3 - DNA, 29 bp, Artificial Sequence
  Description of Artificial Sequence: Synthetic polynucleotide sequence
tttggtggtggtggtggtggtggtggtgg

SEQ ID NO: 4 - DNA, 29 bp, Artificial Sequence
  Description of Artificial Sequence: Synthetic polynucleotide sequence
tttggtggtggtggtttgggtggtggtgg

SEQ ID NO: 5 - DNA, 13 bp, Artificial Sequence
  Description of Artificial Sequence: Synthetic polynucleotide sequence
tggtggtggtggt

SEQ ID NO: 6 - DNA, 14 bp, Artificial Sequence
  Description of Artificial Sequence: Synthetic polynucleotide sequence
ggtggttgtggtgg

SEQ ID NO: 7 - DNA, 15 bp, Artificial Sequence
  Description of Artificial Sequence: Synthetic polynucleotide sequence
gttgtttggggtggt

SEQ ID NO: 8 - DNA, 15 bp, Artificial Sequence
  Description of Artificial Sequence: Synthetic polynucleotide sequence
ttggggggggtgggt

SEQ ID NO: 9 - DNA, 25 bp, Artificial Sequence
  Description of Artificial Sequence: Synthetic polynucleotide sequence
ggttggggtgggtggggtgggtggg

SEQ ID NO: 10 - DNA, 26 bp, Artificial Sequence
  Description of Artificial Sequence: Synthetic polynucleotide sequence
ggtggtggtggttgtggtggtggtgg

SEQ ID NO: 11 - DNA, 26 bp, Artificial Sequence
  Description of Artificial Sequence: Synthetic polynucleotide sequence
ggcggcggcggccgcggcggcggcgg

SEQ ID NO: 12 - DNA, 28 bp, Artificial Sequence
  Description of Artificial Sequence: Synthetic polynucleotide sequence
tttggtggtggtggttgtggtggtggtg

SEQ ID NO: 13 - DNA, 28 bp, Artificial Sequence
  Description of Artificial Sequence: Synthetic polynucleotide sequence
tttggtggtggtggtgtggtggtggtgg

SEQ ID NO: 14 - DNA, 29 bp, Artificial Sequence
  Description of Artificial Sequence: Synthetic polynucleotide sequence
ggtggtggtggttgtggtggtggtggttt

SEQ ID NO: 15 - DNA, 32 bp, Artificial Sequence
  Description of Artificial Sequence: Synthetic polynucleotide sequence
ggtggttgtggtggttgtggtggttgtggtgg

SEQ ID NO: 16 - DNA, 32 bp, Artificial Sequence
  Description of Artificial Sequence: Synthetic polynucleotide sequence
tttggtggtggtggttgtggtggtggtggttt

SEQ ID NO: 17 - DNA, 56 bp, Artificial Sequence
  Description of Artificial Sequence: Synthetic polynucleotide sequence
ggtggtggtggttgtggtggtggtggttgtggtggtggtggttgtggtggtggtgg

SEQ ID NO: 18 - DNA, 35 bp, Artificial Sequence
  Description of Artificial Sequence: Synthetic polynucleotide sequence
tcgagaaaaactctcctctccttccttcctctcca

SEQ ID NO: 19 - DNA, 29 bp, Artificial Sequence
  Description of Artificial Sequence: Synthetic polynucleotide sequence
tttcctcctcctccttctcctcctcctcc

SEQ ID NO: 20 - DNA, 24 bp, Artificial Sequence
  Description of Artificial Sequence: Synthetic polynucleotide sequence
ttagggttagggttagggttaggg

SEQ ID NO: 21 - DNA, 11 bp, Artificial Sequence
  Description of Artificial Sequence: Synthetic polynucleotide sequence
ggtggtggtgg

SEQ ID NO: 22 - DNA, 14 bp, Artificial Sequence
  Description of Artificial Sequence: Synthetic polynucleotide sequence
ggtggttgtggtgg

SEQ ID NO: 23 - DNA, 15 bp, Artificial Sequence
  Description of Artificial Sequence: Synthetic polynucleotide sequence
ggttggtgtggttgg

SEQ ID NO: 24 - DNA, 10 bp, Artificial Sequence
  Description of Artificial Sequence: Synthetic polynucleotide sequence
gggttttggg

SEQ ID NO: 25 - DNA, 20 bp, Artificial Sequence
  Description of Artificial Sequence: Synthetic polynucleotide sequence
ggttttggttttggttttgg

SEQ ID NO: 26 - DNA, 15 bp, Artificial Sequence
  Description of Artificial Sequence: Synthetic polynucleotide sequence
ggttggtgtggttgg

SEQ ID NO: 27 - DNA, 12 bp, Artificial Sequence
  Description of Artificial Sequence: Synthetic polynucleotide sequence
ggggttttgggg

SEQ ID NO: 28 - DNA, 10 bp, Artificial Sequence
  Description of Artificial Sequence: Synthetic polynucleotide sequence
gggttttggg

SEQ ID NO: 29 - DNA, 28 bp, Artificial Sequence
  Description of Artificial Sequence: Synthetic polynucleotide sequence
ggggttttggggttttggggttttgggg

SEQ ID NO: 30 - DNA, 24 bp, Artificial Sequence
  Description of Artificial Sequence: Synthetic polynucleotide sequence
ttggggttggggttggggttgggg

SEQ ID NO: 31 - DNA, 16 bp, Artificial Sequence
  Description of Artificial Sequence: Synthetic polynucleotide sequence
gggtgggtgggtgggt

SEQ ID NO: 32 - DNA, 26 bp, Artificial Sequence
  Description of Artificial Sequence: Synthetic polynucleotide sequence
ggttttggttttggttttggttttgg

SEQ ID NO: 33 - DNA, 29 bp, Artificial Sequence
  Description of Artificial Sequence: Synthetic polynucleotide sequence
tttcctcctcctccttctcctcctcctcc

SEQ ID NO: 34 - DNA, 26 bp, Artificial Sequence
  Description of Artificial Sequence: Synthetic polynucleotide sequence
cctcctcctccttctcctcctcctcc

SEQ ID NO: 35 - DNA, 6 bp, Artificial Sequence
  Description of Artificial Sequence: Synthetic polynucleotide sequence
tggggt

SEQ ID NO: 36 - DNA, 7 bp, Artificial Sequence
  Description of Artificial Sequence: Synthetic polynucleotide sequence
gcatgct

SEQ ID NO: 37 - DNA, 11 bp, Artificial Sequence
  Description of Artificial Sequence: Synthetic polynucleotide sequence
gcggtttgcgg

SEQ ID NO: 38 - DNA, 4 bp, Artificial Sequence
  Description of Artificial Sequence: Synthetic polynucleotide sequence
tagg

SEQ ID NO: 39 - DNA, 23 bp, Artificial Sequence
  Description of Artificial Sequence: Synthetic polynucleotide sequence
ggggttggggtgtggggttgggg

SEQ ID NO: 40-41 - skipped (2 entries)

SEQ ID NO: 42 - DNA, 31 bp, Artificial Sequence
  Description of Artificial Sequence: Synthetic polynucleotide sequence
tgcaggaggtccggcttttccccaacccccc

SEQ ID NO: 43 - DNA, 31 bp, Artificial Sequence
  Description of Artificial Sequence: Synthetic polynucleotide sequence
ggggggttggggaaaagccggacctcctgca

(19) United States
(12) Patent Application Publication
(10) Pub. No.: US 2005/0187176 A1
(43) Pub. Date: Aug. 25, 2005

(54) Method for inhibiting NF-kappa B signaling and use to treat or prevent human diseases

(76) Inventors: Paula J. Bates, Louisville, KY (US); Allicia C. Girvan, Louisville, KY (US); Shirish S. Barve, Louisville, KY (US)

Correspondence Address: EVAN LAW GROUP LLC, 566 WEST ADAMS, SUITE 350, CHICAGO, IL, 60661, US

(21) Appl. No.: 10/961,479

(22) Filed: Oct. 8, 2004

Related U.S. Application Data
(60) Provisional application No. 60/510,466, filed on Oct. 10, 2003.

(51) Int. Cl. A61K 48/00

(52) U.S. Cl. 514/44

(57) ABSTRACT

Methods of treating inflammation in a patient comprise administering to the patient a composition comprising a GRO.

### CROSS REFERENCE TO RELATED APPLICATION

This application claims the benefit of U.S. Provisional Application No. 60/510,466 filed 10 Oct. 2003, which is hereby incorporated by reference in its entirety.

### FEDERALLY SPONSORED RESEARCH OR DEVELOPMENT

The subject matter of this application may have been funded in part by the United States Department of Defense, DAMD17-01-1-0067 (PJB). The government may have certain rights in this invention.

### BACKGROUND

The family of nuclear factor kappa B (NFκB) transcription factors comprises important regulatory proteins that impact virtually every feature of cellular adaptation, including responses to stress, inflammatory reactions, activation of immune cell function, cellular proliferation, programmed cell death (apoptosis), differentiation and oncogenesis (1). NFκB regulates more than 150 genes, including cytokines, chemokines, cell adhesion molecules, and growth factors (2). It is therefore not surprising that diseases result when NFκB-dependent transcription is not appropriately-regulated. NFκB has been implicated in several pathologies, including certain cancers (e.g., Hodgkin's disease, breast cancer, and prostate cancer), diseases associated with inflammation (e.g., rheumatoid arthritis, asthma, inflammatory bowel disease (e.g., Crohn's disease and ulcerative colitis), alcoholic liver disease, non-alcoholic steatohepatitis, pancreatitis, primary dysmenorrhea, psoriasis, and atherosclerosis) and Alzheimer's disease. NFκB is a collective name for dimeric transcription factors comprising the Rel family of DNA-binding proteins (3, 4). All members of this family are characterized by the presence of a conserved protein motif called the Rel homology domain (RHD) that is responsible for dimer formation, nuclear translocation, sequence-specific DNA recognition and interaction with inhibitory proteins collectively known as IκB. Any homodimer or heterodimer combination of family members constitutes NFκB.

Regulation of NFκB Activity

The activity of NFκB is regulated through an assortment of complex signaling pathways, as depicted in FIG. 1. NFκB is negatively regulated through interaction with IκB (5). Each IκB possesses an N-terminal regulatory domain for a signal dependent IκB proteolysis, a domain composed of six or seven ankyrin repeats to mediate interaction with the Rel proteins, and a C-terminal domain containing a PEST motif that is implicated in constitutive IκB turnover. Inactive forms of NFκB reside in the cytoplasm as NFκB/IκB complexes, because IκB binding to NFκB blocks the ability of the nuclear import proteins to recognize and bind to the nuclear localization signal in the RHD.

NFκB activation occurs when NFκB is translocated to the nucleus following its release from IκB. IκB dissociation arises through its phosphorylation by an inducible IκB kinase (IKK) and ubiquitination by IκB ubiquitin ligase, which flags it for proteolysis by the 26S proteosome. Since the ubiquitin ligase and the 26S proteosome are constitutively expressed, the de-repression of NFκB functional activity is largely governed by those signals that induce the expression of IKK, which include inflammatory cytokines, mitogens, viral proteins, and stress.

IKK is also known as the signalsome, which consists of a large multi-subunit complex containing the catalytic subunits IKKα/IKK-1 and IKKβ/IKK-2, a structural subunit termed NFκB essential modulator (NEMO), as well as perhaps other components (6, 7). NEMO, also known as IKKγ and IKKAP-1, functions as an adapter protein to permit communication between the catalytic subunits and upstream activators (7). Activation of NFκB is a tightly controlled process and cannot occur without NEMO (8, 9).

Protein phosphorylation positively regulates NFκB activity (1). Protein phosphorylation enhances the transcriptional activity of NFκB, presumably through the phosphorylated protein's interaction with other transcriptional co-activators. Protein kinase A (PKA), casein kinase II (CKII), and p38 mitogen-activated protein kinase (MAPK) have been implicated in the phosphorylation of NFκB.

The activity of NFκB is also subject to autoregulatory mechanisms to ensure that NFκB-dependent transcription is coordinately-linked to the signal-inducing response. For example, the IκB genes contain NFκB binding sites within their promoter structures that result in their increased transcription upon NFκB binding. The expressed IκB proteins migrate into the nucleus to bind the NFκB and mediate transport of NFκB to the cytoplasm where it remains inactive (1).

Role of NFκB in Disease and Disorders

NFκB contributes to progression of cancers by serving both as positive regulator of cell growth and as a negative regulator of apoptosis (10, 11). NFκB stimulates expression of cell cycle-specific proteins c-Myc and cyclin D1 (12, 13). The constitutive expression of these proteins results in sustained cell proliferation. Continued expression of c-Myc ultimately leads to apoptosis. NFκB can block c-Myc's apoptosis effects, thereby stimulating proliferation without cytotoxicity. NFκB also inhibits the ability of Tumor Necrosis Factor (TNF) to induce cell death as well as protect cells from the effects of ionizing radiation and chemotherapeutic drugs (14). Thus, NFκB promotes both hyperplasia and resistance to oncological treatments, which are hallmarks of many cancers.

Inhibition of NFκB activation has been linked to the chemopreventive properties of several anti-cancer compounds (e.g., selenium, flavonoids, etc.) (15, 16). Although long-term inhibition could have unwanted effects on immune response, down-regulation of NFκB activity is considered a very attractive strategy for developing new cancer treatments. There is currently intense interest in elucidating the details of the signaling pathway (especially the mechanism of NFκB activation) in order to identify suitable molecular targets for therapeutic intervention.

Recently, Shen et al. demonstrated that certain oligonucleotides that contain polyguanonsines are potent inhibitors of the proliferation of murine prostate cancer cells (17). The specific DNA-binding activities of NFκB and another transcription factor, AP-1 were reduced in cells treated with these oligonucleotides. Oligonucleotides displaying antiproliferative effects were capable of forming higher order structures containing guanosine-quartets (G-quartets). The requirement of G-quartets for inducing apoptosis was suggested by experimental observations wherein mutations that destroyed the capacity to form a G-quartet structure correlated with abolishment of the antitumor activities of the oligonucleotide (17).

In the case of inflammation, NFκB plays important roles in both the initiation and maintenance of the inflammatory response (1). Activated T cells, such as activated CD4⁺ T helper cells, trigger immune inflammation. The T helper cell population can differentiate further to two subset populations that have opposite effects on the inflammatory response. The Th1 subset is considered proinflammatory, as these cells mediate cellular immunity and activate macrophages. The Th2 subset is considered anti-inflammatory, as these cells mediate humoral immunity and down-regulate macrophage activation. The subsets are distinguishable by the different types of cytokine profiles that they express upon differentiation. NFκB stimulates production of cytokine profiles characteristic of the Th1 subset type, leading to a proinflammatory response. Conversely, suppression of NFκB activation leads to production of cytokine profiles characteristic of the Th2 subset type that mediates an anti-inflammatory response.

Once activated, these inflammatory cytokines and growth factors can act through autocrine loops to maintain NFκB activation in non-immune cells within the lesion (1). For example, NFκB regulates the expression of cytokines Interleukin 1 beta (IL-1β) and Tumor Necrosis Factor alpha (TNFα), which are considered essential mediators of the inflammatory response. Conversely, these gene products positively activate NFκB expression that leads to persistence of the inflammatory state. For example, TNF products have been implicated in promoting inflammation in several gastrointestinal clinical disorders that include: alcoholic liver disease, non-alcoholic steatohepatitis, prancreatitis (including chronic, acute and alcohol-induced), and inflammatory bowel disorders, such as ulcerative colitis and Crohn's Disease.

Continued NFκB activation also promotes tissue remodeling in the inflammatory lesions (1). Several NFκB-responsive genes have been implicated in this regard and include growth factors that are important to neovascularization (e.g., VEGF), matrix proteinases (including metalloproteases), cyclooxygenase, nitric oxide synthase, and enzymes that are involved in the synthesis of proinflammatory prostaglandins, nitric oxide, and nitric oxide metabolites (1). Such tissue remodeling is often accompanied by breakdown of healthy cells as well as by hyperplasia, both of which are often observed in rheumatoid arthritis and other inflammatory diseases (1).

Suppression of NFκB activity alleviates many inflammatory disease conditions and increases the susceptibility of certain cancers to effective treatment. Several anti-inflammatory drugs directly target the NFκB signaling pathway. Glucocorticoids, one member of the general steroid family of anti-inflammatory drugs, interfere with NFκB function through the interaction of the glucocorticoid receptor with NFκB (18). Gold compounds interfere with the DNA-binding activity of NFκB (19). Aspirin and sodium salicylate, as representatives of non-steroid anti-inflammatory drugs, inhibit IKKβ activity and thereby prevent signal-inducible IκB turnover (20). Dietary supplements with anti-inflammatory and anti-tumor activities prevent NFκB activation by interfering with pathways leading to IKK activation. Vitamins C and E, prostaglandins, and other antioxidants, scavenge reactive oxygen species that are required for NFκB activation (21, 22). Specific NFκB decoys that mimic natural NFκB ligands (e.g., synthetic double-stranded oligodeoxynucleotides that contain the NFκB binding site) can suppress NFκB activity and prevent recurrent arthritis in animal models (23).

Despite the promise of anti-inflammatory drugs in treating inflammatory diseases, many diseases are non-responsive to these modalities. For example, many patients with chronic inflammatory diseases, such as Crohn's disease, fail to respond to steroid treatment. Recent studies suggest that one basis for the steroid unresponsiveness may be attributed to NFκB and other NFκB-responsive gene products antagonizing glucocorticoid receptor expression, which is necessary for the steroid's anti-inflammatory activity (24).

Alzheimer's disease represents another example of a condition that displays an inflammatory component in its pathogenesis. Recent studies indicate that abnormal regulation of the NFκB pathway may be central to the pathogenesis of Alzheimer's disease. NFκB activation correlates with the initiation of neuritic plaques and neuronal apoptosis during the early phases of the disease. For example, NFκB immunoreactivity is found predominantly in and around early neuritic plaque types, whereas mature plaque types display reduced NFκB activity (25).

Inflammation is a prevalent component in many diseases and disorders, such as: cancer, both acute and chronic inflammation, gastrointestinal tract disorders and Alzheimer's disease. The NFκB signaling pathway plays a pivotal role in coordinating genes involved in the inflammatory response. The present invention proposes a novel set of methods and compositions that interfere with the NFκB signaling pathway to disrupt NFκB-mediated regulation of these genes as a means of reducing inflammation that is associated with many diseases and disorders.

### SUMMARY

In a first aspect, the present invention is a method of treating inflammation in a patient, comprising administering to the patient a composition comprising a GRO.

In a second aspect, the present invention is a pharmaceutical composition comprising an amount of a GRO effective for inflammation therapy, an anti-inflammatory agent, and a pharmaceutically acceptable carrier.

In a third aspect, the present invention is a method for providing chronic inflammation therapy to a mammal, comprising administering an effective amount of a pharmaceutical composition, comprising a vesicle, an anti-inflammatory agent, and a pharmaceutically acceptable carrier. The vesicle comprises an amount of a GRO effective for inflammation therapy.

In a fourth aspect, the present invention is a method for determining the efficacy of treating inflammation with a GRO, comprising administering the GRO; and measuring a change in an NFκB activity before and after administration of the GRO.

In a fifth aspect, the present invention is a pharmaceutical composition comprising an amount of a GRO effective for inflammation therapy and a pharmaceutically acceptable carrier. The pharmaceutical composition is supplied as one selected from the group consisting of a suppository, a cream, an enema, and an aerosol.

Definitions

The phrase “G-quartet” refers to an arrangement of four guanosines that form a planar hydrogen-bonded structure wherein the guanosines are believed to engage in Hoogstein hydrogen-bonding, reverse Hoogstein hydrogen-bonding, or a combination of both hydrogen bonding schemes.

The phrase “G-rich oligonucleotide” refers to an oligonucleotide that contains greater representation of guanosines than adenosines, thymidines, or cytosines.

The term “GRO” refers to a particular class of G-rich oligonucleotides.

Characteristics of GROs include:

(1) preferably having at least 1 GGT motif,
(2) having at least four GG dinucleotides,
(3) preferably having 9-100 nucleotides, although GROs having many more nucleotides are possible; more preferably having at least 24 nucleotides, and
(4) displaying the propensity to form a G-quartet structure having at least two stacked G-quartets involving at least four GG dinucleotides.

### BRIEF DESCRIPTION OF THE FIGURES

FIG. 1 depicts the NFκB signaling pathway.

FIG. 2 depicts the structure of a G-quartet (A) and a schematic representation of GRO26B [SEQ ID NO:10] that contains 8 G-quartets (B).

FIG. 3 depicts phase contrast (A) and fluorescent (B) images showing the uptake of FITC-GRO26B into lung cancer cells in the absence (upper panels) and presence (lower panels) of nucleolin antibody.

FIG. 4 depicts the effect of GRO26B on tumor growth (A) and final tumor volume (B) in a DU145 prostate cancer xenograft model.

FIG. 5 depicts a silver-stained gel of specific proteins that precipitate with a biotinylated GRO29A oligonucleotide following strepavidin bead selection (lower panel).

FIG. 6 depicts the results of a Western blot experiment that demonstrates that nucleolin and NEMO are associated with GRO in cells treated with GRO.

FIG. 7 depicts the results of a Western blot experiment that demonstrates NEMO is associated with both nucleolin and GRO in GRO-treated cells.

FIG. 8 illustrates that GRO specifically inhibits DNA-binding by NFκB (A) and inhibits NFκB-mediated activation of gene expression, as detected by luciferase activity (B).

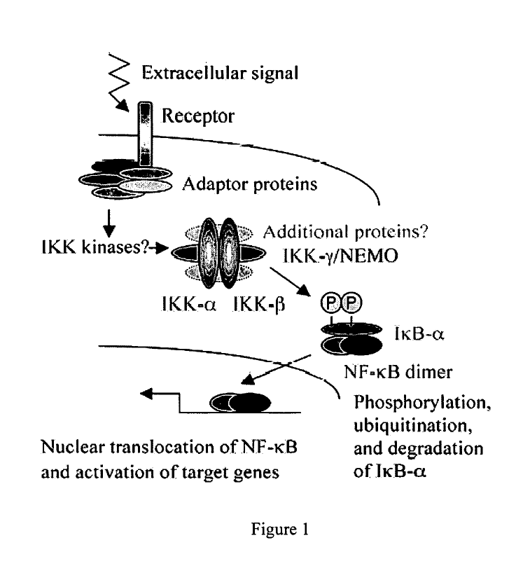

### DETAILED DESCRIPTION

The present invention makes use of the discovery of G-rich oligonucleotides as a new class of antiproliferative, pro-apoptotic, and anti-inflammatory agents that have tremendous therapeutic potential for cancer treatment and disease conditions that have an inflammatory component as part of their pathology. These G-rich oligonucleotides (GROs) interfere with the NFκB signaling pathway by blocking NEMO function of IKK. Thus, GROs work by a mechanism that is completely different from any known chemotherapy/anti-inflammatory agent or antisense oligonucleotide. The unusual structure of GROs confers properties different from those expected for an unmodified DNA oligonucleotide. These include enhanced cellular uptake as naked DNA, extreme thermal stability, and nuclease resistance, thereby making them ideal for therapeutic uses. They are selective for malignant cells and active in vivo against tumors. Furthermore, GROs act as anti-inflammatory agents to reduce the symptoms associated with inflammatory diseases.

The present invention was discovered during investigations that employ GROs designed for triple helix formation and their unexpected ability to effect antiproliferation of cultured prostate carcinoma cells (26). The antiproliferative effects were not consistent with a triplex-mediated or an antisense mechanism, and it was apparent that certain GROs were inhibiting proliferation by an alternative mode of action. It was surmised that GROs, which display the propensity to form higher order structures containing G-quartets, work by an aptamer mechanism that entails binding to specific cellular proteins due to a shape-specific recognition of the GRO structure.

Preferred GROs include those oligonucleotides that can form the G-quartet structure having two or fewer independent oligonucleotides. Such GROs are preferred because they share a propensity to form a G-quartet structure under entropically-favored conditions. Preferred GROs include those that form the G-quartet structure from two separate oligonucleotide strands. These GROs may display one of two types of dimer quadraplex conformations: a dimer hairpin chair conformation or a dimer hairpin basket conformation.

Even more preferred GROs are those oligonucleotides that can form G-quartet structures from one oligonucleotide strand. Such GROs are more preferred because they should form a G-quartet structure independent of their concentration in solution. Such GROs may adopt two types of monomer quadraplex conformations: a monomer chair conformation or a monomer basket conformation.

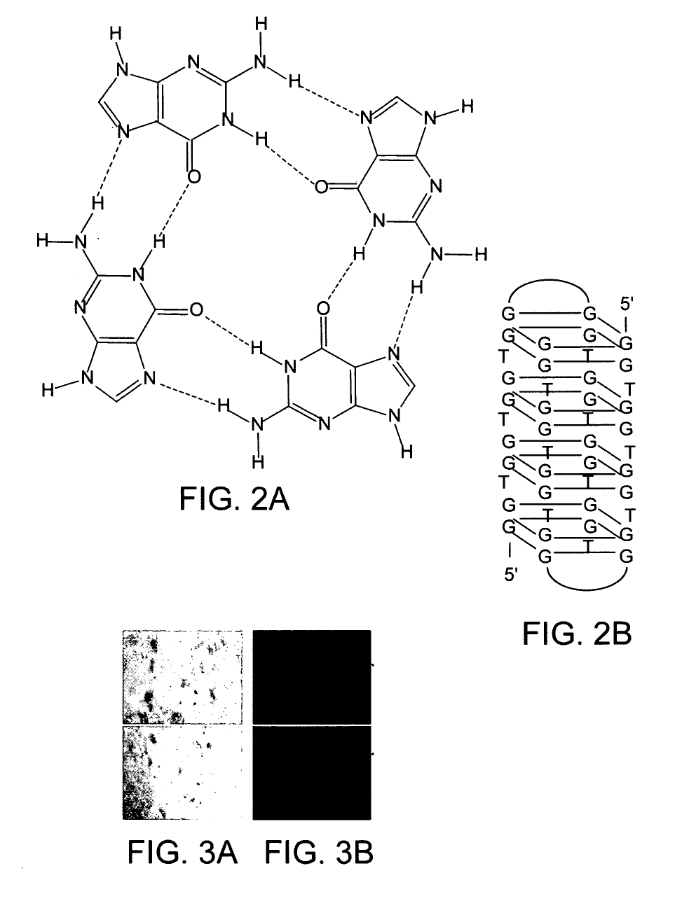

The original GRO (GRO29A [SEQ ID NO:1]) is a synthetic oligonucleotide with a phosphodiester DNA backbone, whose sequence is 5′-TTTGGTGGTGGTGGTTGTGGTGGTGGTGGX-′3, where the X is a propylamine group. The 5′-TTT leader and 3′-terminal modification are not necessary for optimal activity, and the preferred GRO is GRO26B [SEQ ID NO:10] 5′-GGTGGTGGTGGTTGTGGTGGTGGTGG-′3. Molecular modeling studies indicate the preferred structure for this sequence is a hairpin dimer quadruplex containing two folded strands that are stabilized by a total of eight G-quartets (FIG. 2). The unusual structure of GRO26B confers properties different from those expected for an unmodified DNA oligonucleotide. These include enhanced cellular uptake as naked DNA (e.g., see FIG. 3), extreme thermal stability (e.g., Tₘ≈76° C.), and nuclease resistance (e.g., no degradation after 5 days in serum-containing medium).

GROs can bind to the cellular protein nucleolin. This protein is highly expressed in proliferating cells but is undetectable in quiescent cells (27). Furthermore, there is a strong, positive correlation between nucleolin protein levels and the rate of cell proliferation (27). These relationships are used in oncology to detect silver staining nucleolar organizer region proteins, of which nucleolin is a major and constant component (28). For example, levels of these proteins have been demonstrated to correlate with tumor doubling time and reliably predict clinical outcome in a variety of cancers (29). Importantly, increases in the level of this protein are also associated with progression through specific stages of cancer, i.e., from normal to pre-malignant to malignant states, during chemical induced carcinogenesis in rats, and during the development of hepatocellular carcinoma in humans (30). The chromosomal region containing the nucleolin gene is frequently duplicated or translocated in a broad spectrum of adenocarcinomas, leukemias, and lymphomas (31). High levels of nucleolin correlate with malignant disease.

GROs were originally characterized for their ability to bind to nucleolin, as well as their utility as a method for detecting malignant diseases, like prostate cancer. This and other methods for use of GROs are described in U.S. patent application Ser. No. 10/118,854, titled “METHOD FOR THE DIAGNOSIS AND PROGNOSIS OF MALIGNANT DISEASES,” to Paula J. Bates et al., filed on Apr. 8, 2002, the contents which are hereby incorporated by reference in its entirety.

The ability of GROs to bind nucleolin suggested that their antiproliferative effects are attributed to the ability to alter one or more activities of nucleolin. The identification of the affected function(s) of this protein is daunting, as the protein is large (e.g., the human protein has 707 amino acids), is composed of multi-domain structures, and is implicated in diverse cellular roles, such as ribosome biogenesis, DNA replication, cell cycle progression, stress response, protein transport, and apoptosis (32-38). Although considered as a nucleolar protein, nucleolin is also found in the nucleoplasm, cytoplasm, and on the plasma membrane surface as a receptor (26, 39-44). The expression of plasma membrane-associated nucleolin is most often seen in neoplastic cells (such as malignant or pre-malignant) (38). In addition, a correlation between nucleolin plasma membrane expression and the aggressiveness of neoplastic disease has been identified (38).

Given the intracellular mobility of the protein, nucleolin may serve as a cellular matchmaker in the interactions between proteins and their targets. That is, nucleolin may be involved in the transport of proteins to their appropriate targets and also in the modulation of their processing activity. The present invention contemplates this mechanism, as one of many, as the basis for the ability of GRO to interfere with nucleolin activity and to mediate its antiproliferative effects on cancer cells and its anti-inflammatory effects in diseases that have inflammation as a component of their pathogeneses.

Although it was previously unrecognized that nucleolin is involved in NFκB regulation, it was realized that many of the same stimuli that activate NFκB (e.g., ligation of CD21, PKC-ζ, or T cell receptors, viral infection, lipopolysaccharide glycosylated proteins, and UV light) have been linked to nucleolin binding or mobilization (38, 39, 45-53). For example, both NEMO and nucleolin are known to associate with the activated T cell receptor complex (50, 51). It was therefore of interest to ascertain whether GRO-nucleolin interactions affect NFκB regulation.

In the instant invention, it is shown that GRO-nucleolin interactions adversely affect NFκB regulation. NFκB signaling is blocked in cells after their treatment with GROs. Furthermore, GROs blocked TNFα-stimulated NFκB transcriptional activity of a luciferase reporter gene linked to a NFκB-responsive promoter. The failure of TNFα-stimulated NFκB transcriptional activity in the presence of GROs is attributed to the unavailability of NFκB to bind to its consensus DNA target. Finally, it has been discovered that nucleolin is stably associated with both NEMO and GRO in cells treated with GROs. None of these effects is observed for cells treated with control oligonucleotides lacking the capacity to form G-quartets or with control buffers lacking oligonucleotide.

The mechanisms whereby GROs exert their effects on the NFκB signaling pathway are not completely understood, and the invention is not limited to any particular mode of GRO function in this regard. As one possible mechanism, GRO-mediated inhibition of cellular proliferation may be partly attributed to the ability of GRO-associated nucleolin to interfere with the NEMO activity of IKK. Nucleolin associates with NEMO during normal NFκB signaling, perhaps serving to present the NEMO-responsive signal to NEMO to stimulate IKK activation. GRO may represent a signal decoy antagonist that fails to trigger NEMO's normal activity. Alternatively, GRO may induce within nucleolin an alternate binding conformation specific for NEMO that prevents presentation of an appropriate signal necessary for IKK activation. Regardless of the precise details of the GRO-nucleolin-NEMO ternary interaction, the complex appears to interfere with the NFκB signaling pathway involving the activity of IKK wherein NFκB remains unable to dissociate from IκB in the cytoplasm.

The present invention contemplates the use of GROs in the treatment of inflammatory diseases, and disorders that possess an inflammatory component of their pathology, such as Alzheimer's disease and a variety of gastrointestinal disorders. By disrupting the NFκB signaling pathway mediated by NEMO, GROs have therapeutic utility in the treatment of these conditions by reducing the expression of genes regulated by NFκB that contributes to the inflammatory response.

Inflammatory diseases include those associated with acute inflammation as well as chronic inflammation. Examples of an acute inflammation that the present invention may be used for include acute inflammatory conditions characterized by rapid onset, such as: dental pain, head pain, generalized joint pain, acute pancreatitis, and primary dysmenorrhea. Primary dysmenorrhea is attributed to uterine contractions that arise during menstrual periods in up to 90% of women, resulting in cramps that are frequent, intense, and severe. Examples of a chronic inflammation that the present invention may also be used for include those associated with chronic inflammatory diseases with progressive, delayed or slow onset, such as: rheumatoid arthritis, asthma, psoriasis, atherosclerosis and gastrointestinal tract disorders, such as inflammatory bowel disease (e.g., Crohn's disease and ulcerative colitis), alcoholic liver disease, non-alcoholic steatohepatitis, chronic pancreatitis, and alcohol-induced pancreatitis.

The inflammatory component of Alzheimer's disease may also be treated with GROs. An increase in NFκB expression has been implicated in the initiation of neuritic plaques and neuronal apoptosis during the early phases of Alzheimer's disease. Suppression of NFκB activity by administration of GROs would alleviate some of the symptoms associated with this disease. Familial history of a genetic predisposition to the disease may be instrumental for identifying individuals for treatment with GROs to reduce the likelihood or severity of disease symptoms. In this regard, early detection of disease predisposition or disease onset would be advantageous for use of the present invention in effective treatment.

Since the resistance of many inflammatory diseases to the activity of anti-inflammatory drugs is attributed to NFκB, suppression of NFκB activity would alleviate many inflammatory disease conditions or render them more susceptible to treatment with anti-inflammatory agents. Although several anti-inflammatory drugs are known to directly target the NFκB signaling pathway, GROs represents a novel means for inhibiting this pathway. The usefulness of GROs to specifically interfere with NEMO function has at least three important therapeutic advantages in the treatment of inflammatory diseases. First, GROs may alter the balance of immune cell subset function associated with inflammatory responses, favoring production of cytokine profiles characteristic of the Th2 subset type that mediates an anti-inflammatory response and permitting a reduction in inflammation. Second, GROs may short-circuit autocrine loops at work in pre-existing inflammatory lesions, resulting in de-amplification of NFκB-activated gene expression that is responsible for sustaining inflammation in non-immune cells, thereby attenuating inflammation. Third, GROs may be used as a prophylactic agent to prevent recurrence of the underlying inflammatory conditions that are associated with acute pain and inflammation (e.g., primary dysmenorrhea) and that which is associated with chronic inflammation (e.g., rheumatoid arthritis).

The present invention are not limited to humans, as other species also suffer inflammation. For example, dogs and cats suffer inflammatory bowel disease, which includes the symptoms of diarrhea and vomiting. Thus, the present invention includes treatments of inflammation for a diverse number of species, including mammalian species. Examples of mammalian species contemplated as patients of GRO treatments include: humans, dogs, cats, cattle, sheep, goats, horses, buffalo, and pigs. Avians and amphibians are also contemplated as patients.

The present invention may use GROs in combination modalities for the treatment of diseases that possess an inflammatory component to their pathologies, such as acute inflammation, chronic inflammation, and Alzheimer's disease. In this regard, the present invention includes combination compositions wherein another anti-inflammatory agent and a GRO may promote synergistic reduction of the inflammatory state. Examples of anti-inflammatory agents that are useful for this purpose include: glucocorticosteroids, nonsteroidal anti-inflammatory agents, flavonoids, vitamin A, vitamin C, cyclopentenone prostaglandins, tacrolimus, cyclosporin A, etc. Preferred corticosteroids include: dexamethasone, hydrocortisone, triamcinolone acetonide, clobetasol propionate, flurandrenolide fluocinolone acetonide prednicarbate and triamcinolone acetonide. Preferred nonsteroidal anti-inflammatory agents include: aspirin, ibuprofen, naproxen, nabumetone, and the like. An example of one preferred flavonoid is resveratrol. The resveratrol may be derived synthetically or from natural sources, such as red wine.

Anti-inflammatory agents may exert their influence by interfering with the NFκB signaling pathway. Preferred anti-inflammatory agents include those that act at different steps in the NFκB signaling pathway than those postulated for GROs. For example, aspirin, a non-steroid anti-inflammatory drug, inhibits IKKβ activity and thereby prevents signal-inducible IκB turnover.

Anti-inflammatory agents may also manifest their activity by interfering with the action of the products of inflammatory genes whose expression is increased by NFκB. Examples of genes that NFκB regulates include those listed in Table 1. Inhibition of prostaglandin biosynthesis is one preferred approach for treating inflammation, since prostaglandins contribute to the inflammatory condition. The COX-2 enzyme represents one preferred target of an anti-inflammatory agent owing to its role in the synthesis of prostaglandins. Preferred COX-2 enzyme inhibitors include celecoxib, rofecoxib, and valdecoxib, which are the generic formulations of CELEBREX®, VIOXX®, and BEXTRA®, respectively.

Another attractive target for treating inflammation is to target the function of another NFκB-regulated gene, TNF. As described previously, TNF is one of the NFκB-regulated genes (see Table I) and the protein also stimulates NFκB expresssion. This naturally occurring cytokine plays an important role in the inflammatory processes of rheumatoid arthritis (RA), polyarticular-course juvenile rheumatoid arthritis (JRA), and ankylosing spondylitis and the resulting joint pathology. Elevated levels of TNF are found in involved tissues and fluids of patients with RA, psoriatic arthritis and ankylosing spondylitis. Two distinct receptors for TNF (TNFRs), a 55 kilodalton protein (p55) and a 75 kilodalton protein (p75), exist naturally as monomeric molecules on cell surfaces and in soluble forms. Biological activity of TNF is dependent upon binding to either cell surface TNFR. Thus, drugs that block the interaction between TNF with TNFRs will serve as an effective means for inhibiting NFκB expression and indirectly the NFκB signaling pathway.

Etanercept, which is the generic form of ENBREL®, binds specifically to TNF and blocks its interaction with cell surface TNFRs. Etanercept is a dimeric soluble form of the p75 TNFR that can bind to two TNF molecules. Etanercept inhibits binding of both TNFα and TNFβ to cell surface TNFRs, rendering TNF biologically inactive. In this manner, etanercept can modulate the biological responses induced or regulated by TNF, including gene expression programs of the NFκB signaling pathway. Thus, combinations of GROs with TNF function inhibitors like etanercept may be used effectively treat conditions that contain an inflammatory component as part of their pathology. The aforementioned gastrointestinal tract clinical disorders represent attractive targets for the use such combination therapies involving GROs and TNF function inhibitors, as one manifestation of these disorders, such as alcholic liver disease and pancreatitis, is positively-disregulated TNF and NFκB activities.

The efficacy of GROs in treating conditions or diseases associated with inflammation may be evaluated by monitoring the inhibition of the NFκB signaling pathway in patients following administration of GRO-containing compositions. NFκB inactivation may be inferred by its inability to undergo nuclear translocation, as revealed with the use of an electrophoretic mobility shift assay, ELISA, or immunochemistry. GRO-mediated inhibition of the NFκB signaling pathway may be inferred by monitoring the reduction of NFκB-mediated gene expression. Since NFκB regulates over 150 genes (Table 1), suitable molecular tools may be chosen to monitor any of their expression profiles.

TABLE 1
NFκB-responsive genes¹

| Gene | Function |
|---|---|
| CINC | Cytokine-induced neutrophil chemoattractant |
| Eotaxin | β Chemokine, eosinphil-specific |
| Gro α-γ | Melanoma growth stimulating activity |
| IFN-γ | Interferon |
| IL-1α | Interleukin-1α |
| IL-1β | Interleukin-1β |
| IL-1-receptor antagonist | Inhibitor of IL-1 activity |
| IL-2 | Interleukin-2 |
| IL-6 | Interleukin-6, inflammatory cytokine |
| IL-8 | Interleukin-8, α-chemokine |
| IL-11 | Interleukin-11 |
| IL-12 (p40) | Interleukin-12 |
| β-Interferon | Interferon |
| IP-10 | α Chemokine |
| KC | α Chemokine |
| Lymphotoxin β | Anchors TNF to cell surface |
| MCP-1/JE | Macrophage chemotactic protein, β Chemokine |
| MIP-1α,β | Macrophage inflammatory protein-1, β Chemokine |
| MIP-2 | Macrophage inflammatory protein-1, β Chemokine |
| RANTES | Regulated upon Activation Normal T lymphocyte |
| | Expressed and Secreted, β Chemokine |
| TCA3, T-cell activation gene 3 | T-cell activation gene 3, β Chemokine |
| TNFα | Tumor necrosis factor α |
| TNFβ | Tumor necrosis factor β |
| B7.1 (CD80) | Co-stimulation of T cells via CD28 binding |
| BRL-1 | B-cell homing receptor |
| CCR5 | Chemokine receptor |
| CD48 | Antigen of stimulated lymphocytes |
| F_{c} epsilon receptor II (CD23) | Receptor for IgE |
| IL-2 receptor α-chain | IL-2 receptor subunit |
| Immunoglobulin Cγ1 | IgG heavy chain |
| Immunoglobulin ε heavy chain | IgE heavy chain |
| Immunoglobulin κ light chain | Antibody light chain |
| Invariant Chain II | Antigen presentation |
| MHC class I (H-2K^{b}) | Mouse histocompatibility antigen |
| MHC Class I HLA-B7 | Mouse histocompatibility antigen |
| β2 Microglobulin | Binds MHC class I |
| T-cell receptor β chain | T-cell receptor subunit |
| Proteasome Subunit LMP2 | Subunit of 26S proteosome, cysteine protease |
| Peptide Transporter TAP1 | Peptide transporter for ER |
| ELAM-1 | E-selectin, endothelial cell leukocyte adhesion |
| | molecule |
| ICAM-1 | Intracellular adhesion molecule-1 |
| MadCAM-1 | Mucosal addressin cell adhesion molecule |
| P-selectin | Platelet adhesion receptor |
| Tenascin-C | ECM protein controls cell attachment and migration, |
| | cell growth |
| VCAM-1 | Vascular cell adhesion molecule |
| Angiotensinogen | Angiotensin precursor, regulates blood pressure |
| C4b binding protein | Complement binding protein |
| Complement factor B | Complement factor |
| Complement Factor C4 | Activates extrinsic pathway of complement activation |
| C-reactive protein | Pentraxin |
| LPS binding protein | Binds to LPS receptor (CD14) with LPS |
| Pentraxin PTX3 | Pentraxin |
| Serum amyloid A precursor | Serum component |
| Tissue factor-1 | Activates extrinsic pathway of complement activation |
| Urokinase-type Plasminogen | Activates fibrinogen for fibrin clot lysis |
| activator | |
| Angiotensin II | Peptide hormone |
| COX-2 [SEQ ID NOs: 40 and 41] | Cyclooxygenase, prostaglandin endoperoxide synthase |
| Ferritin H chain | Iron storage protein |
| 12-Lipoxygenase | Arachidonic acid metabolic enzyme |
| inducible NO-Synthase | NO synthesis |
| Mn SOD | Superoxide dismutase |
| NAD(P)H quinone | Bioreductive enzyme |
| oxidoreductase (DT-diaphorase) | |
| Phospholipase A2 | Fatty acid metabolism |
| A1 adenosine receptor | Pleiotropic physiological effects |
| Bradikinin B1-Receptor | Pleiotropic physiological effects |
| CD69 | Lectin mainly on activated T cells |
| Gall Receptor | Galanine receptor, neuroendocrine peptide |
| Lox-1 | Receptor for Oxidized low density lipoprotein |
| Mdr1 | Multiple drug resistance mediator (P-glycoprotein) |
| Neuropeptide Y Y1-receptor | Pleiotropic physiological effects |
| PAF receptor 1 | Platelet activator receptor |
| RAGE-receptor for advanced | Receptor for Advanced Glycation End products |
| glycation end products | |
| Bfl1/A1 | Pro-survival Bcl-2 homologue |
| Bcl-xL | Pro-survival Bcl-2 homologue |
| Nr13 | Pro-survival Bcl-2 homologue |
| cCD95 (Fas) | Pro-apoptotic receptor |
| Fas-Ligand | Inducer of apoptosis |
| IAPs | Inhibitors of Apoptosis |
| IEX-1L | Immediate early gene |
| G-CSF | Granulocyte Colony Stimulating Factor |
| GM-CSF | Granulocyte Macrophage Colony Stimulating Factor |
| IGFBP-2 | Insulin-like growth factor binding protein-2 |
| M-CSF (CSF-1) | Macrophage Colony Stimulating Factor |
| PDGF B chain | Platelet-Derived Growth Factor |
| Proenkephalin | Hormone |
| VEGF C | Vascular Endothelial Growth Factor |
| p22/PRG1 | Rat homology of IEX |
| p62 | Non-proteosomal multi-ubiquitin chain binding |
| | protein |
| A20 | TNF-inducible zinc finger |
| c-myb | Proto-oncogene |
| c-myc | Proto-oncogene |
| c-rel | Proto-oncogene |
| IRF-1 | Interferon regulatory factor-1 |
| IRF-2 | Interferon regulatory factor-2 |
| IκBα | Inhibitor of Rel/NF-κB |
| junB | Proto-oncogene |
| nfkb2 | NF-κB p100 precursor |
| nfkb1 | NF-κB p105 precursor |
| p53 | Tumor suppressor |
| Collagenase 1 | Matrix metalloproteinase |
| Gelatinase B | Matrix metalloproteinase |
| GSTP1-1 | Glutathione transferase |
| Glucose 1-6-phosphate | Hexose monophosphate |
| dehydrogenase | |
| Hyaluronan synthase | Synthesizes hyaluronic acid |
| Lysozyme | Hydrolyzes bacterial cell walls |
| Transglutaminase | Forms isopeptide bonds |
| alpha-1 acid glycoprotein | Serum protein |
| Apolipoprotein C III | Apoprotein of HDL |
| Cyclin D1 | Cell-cycle regulation |
| Factor VIII | Hemostasis |
| Galectin 3 | β-galactosidase-binding lectin |
| HMG14 | High mobility group 14 |
| K3 Keratin | Intermediate filament protein |
| Laminin B2 Chain | Basement membrane protein |
| Mts1 | Multiple tumor suppressor |
| Vimentin | Intermediate filament protein |
| α1-antitrypsin | Protease inhibitor |

¹Adapted from ref. #2.

Suitable molecular tools include: nucleic acid sequences specific for the affected genes and/or their transcription products; translation products of the affected genes or suitable polypeptide derivatives thereof; and antibodies specific for the translation products of affected genes or suitable polypeptide fragments thereof. Nucleic acids, polypeptides, and polypeptide-specific antibodies may be prepared for these purposes using methods readily available to the skilled artisan.

A nucleic acid probe is any nucleic acid sequence that displays the ability to hybridize to a desired target DNA or RNA sequence via base-specific complementarity. A nucleic acid probe in the present invention is any sequence having at least ten nucleotides or the complement thereof. More preferably, a nucleic acid probe is any sequence having at least 15, 20, 25, 30, 50, or 100 nucleotides, or the complement thereof. Even more preferably, a nucleic acid probe is any sequence encompassing the entire length of the target gene, or the complement thereof. A nucleic acid probe may be used in either a labeled or an unlabeled form. Probes can be labeled at the 5′ and/or 3′ terminus or at internal positions. Examples of labels include radioactive groups (e.g., ³H and ³²P) and dyes that display chromogenic properties, such as fluorescence or phosphorescence.

Solid phase support matrices may be used to monitor the expression of the affected genes. Optionally, nucleic acid arrays may be employed to monitor gene expression profiles, wherein the particular nucleic acid is immobilized to a solid phase support and hybridized with a nucleic acid probe specific for a particular sequence. Such arrays offer an opportunity to simultaneously monitor expression profiles for several independently-regulated genes.

A polypeptide suitable for the present invention is any amino acid sequence that binds to a GRO, that binds to another polypeptide with GRO-binding activity, or whose epigenetic or genetic activity is affected by a GRO. Preferred polypeptides include any amino acid sequence to which an antibody may be generated that binds to NEMO, nucleolin, NFκB, or translation products of genes whose activity is regulated by NFκB. Preferred polypeptides include those with at least ten amino acids. More preferably, polypeptides of 10, 15, 20, 20, 30, 40, 50, 75, and 100 amino acids are contemplated in the present invention. Most preferably, polypeptides that span the entire open reading frame of the gene of interest are contemplated in the present invention. Polypeptides for use in the present invention may be labeled either during their synthesis or following their isolation and purification. Suitable labels include radioactive groups (e.g., ³⁵S, ³H, ¹⁴C, ¹²⁵I) and dyes that display chromogenic properties, such as fluorescence or phosphorescence.

An antibody suitable for the present invention includes any antibody of whatever structure and prepared by whatever means available in the art, including monoclonal, polyclonal, hybrid, or single-chain antibodies. Preferred antibodies include those that bind to a polypeptide that displays GRO-binding activity, interacts with a GRO-binding polypeptide or whose epigenetic or genetic activity is affected by a GRO. More preferably, the present invention includes use of an antibody that binds to NEMO, nucleolin, NFκB, or translation products of genes whose activity is regulated by NFκB or polypeptide fragments thereof. Such antibodies may be generated using as antigen polypeptides that have at least ten amino acids. More preferably, the present invention includes antibodies generated using as antigen polypeptides of 10, 15, 20, 20, 30, 40, 50, 75, and 100 amino acids. Most preferably, the present invention contemplates antibodies generated with polypeptides that span the entire open reading frame of the affected gene of interest. Antibodies for use in the present invention may be labeled either during their synthesis or following their isolation and purification. Suitable labels include radioactive groups (e.g., ³⁵S, ³H, ¹⁴C, ¹²⁵I) and dyes that display chromogenic properties, such as fluorescence or phosphorescence.

Long-term administration of GROs may result in development of an immune response to the molecules that mediate GRO clearance from the patient. Thus, administration regimens that include successive use of GROs differing in their structure will avoid immunologic clearance of specific GROs. More preferably, encapsulation of GROs in appropriate carriers, such as in particles, capsules, or tablets may circumvent clearance of GROs, thereby rendering them more effective agents for long-term treatments of diseases or conditions associated with inflammation.

Examples of GROs useful for the present invention are illustrated in Table 2. Preferred GROs display nucleolin-binding activity, as assessed by a number of techniques. For example, preferred GROs compete with a telomere oligonucleotide for binding to nucleolin in an electrophoretic mobility shift assay (26). Preferred GROs form G-quartet structures, as indicated by a reversible thermal denaturation/renaturation profile at 295 nm (26). Telomere oligonucleotides also form stable G-quartet structures, thereby providing adequate guidance as to the design of GROs as structural candidate sequences of the present invention. More preferably, GROs that form GRO-nucleolin-NEMO ternary complexes are contemplated in the present invention. Such complexes may be discerned using a variety of methods, such as those described in Example 3. Even more preferably, GROs that inhibit the NFκB signaling pathway are contemplated in the present invention, as judged by a GRO's ability to inhibit IKK activity (e.g., Example 4). Examples of preferred GROs include those with nucleic acid sequences of SEQ ID NOs: 1, 10, 11, and 26-32.

TABLE 2

Examples of GRO and non-GRO nucleic
acids^{1,2,3}

| | | |
|---|---|---|
| | | SEQ ID |
| GRO | Sequence | NO: |

| | | |
|---|---|---|
| GRO29A¹ | tttggtggtg gtggttgtgg tggtggtgg | 1 |
| GRO29-2 | tttggtggtg gtggttttgg tggtggtgg | 2 |
| GRO29-3 | tttggtggtg gtggtggtgg tggtggtgg | 3 |
| GRO29-5 | tttggtggtg gtggtttggg tggtggtgg | 4 |
| GRO29-13 | tggtggtggt ggt | 5 |
| GRO14C | ggtggttgtg gtgg | 6 |
| GRO15A | gttgtttggg gtggt | 7 |
| GRO15B² | ttgggggggg tgggt | 8 |
| GRO25A | ggttggggtg ggtggggtgg gtggg | 9 |
| GRO26B¹ | ggtggtggtg gttgtggtgg tggtgg | 10 |
| GRO26C | ggcggcggcg gccgcggcgg cggcgg | 11 |
| GRO28A | tttggtggtg gtggttgtgg tggtggtg | 12 |
| GRO28B | tttggtggtg gtggtgtggt ggtggtgg | 13 |
| GRO29-6 | ggtggtggtg gttgtggtgg tggtggttt | 14 |
| GRO32A | ggtggttgtg gtggttgtgg tggttgtggt gg | 15 |
| GRO32B | tttggtggtg gtggttgtgg tggtggtggt tt | 16 |
| GRO56A | ggtggtggtg gttgtggtgg tggtggttgt | 17 |
| | ggtggtggtg gttgtggtgg tggtgg | |
| CRO1 | tcgagaaaaa ctctcctctc cttccttcct | 18 |
| | ctcca | |
| CRO2 | tttcctcctc ctccttctcc tcctcctcc | 19 |
| GRO A | ttagggttag ggttagggtt aggg | 20 |
| GRO B | ggtggtggtg g | 21 |
| GRO C | ggtggttgtg gtgg | 22 |
| GRO D | ggttggtgtg gttgg | 23 |
| GRO E | gggttttggg | 24 |
| GRO F | ggttttggtt ttggttttgg | 25 |
| GRO G¹ | ggttggtgtg gttgg | 26 |
| GRO H¹ | ggggttttgg gg | 27 |
| GRO I¹ | gggttttggg | 28 |
| GRO J¹ | ggggttttgg ggttttgggg ttttgggg | 29 |
| GRO K¹ | ttggggttgg ggttggggtt gggg | 30 |
| GRO L¹ | gggtgggtgg gtgggt | 31 |
| GRO M¹ | ggttttggtt ttggttttgg ttttgg | 32 |
| GRO N² | tttcctcctc ctccttctcc tcctcctcc | 33 |
| GRO O² | cctcctcctc cttctcctcc tcctcc | 34 |
| GRO P² | tggggt | 35 |
| GRO Q² | gcatgct | 36 |
| GRO R² | gcggtttgcg g | 37 |
| GRO S² | tagg | 38 |
| GRO T | ggggttgggg tgtggggttg ggg | 39 |

¹Indicates a good nucleolin-binding GRO.
²Indicates a nucleolin control (non-plasma membrane nucleolin binding).
³GRO sequence without 1 or 2 designations have some anti-proliferative activity.

GROs may be prepared using conventional oligonucleotide synthesis procedures, such as phosphoramidite triester methods. Those GRO aptamers capable of forming stable G-quartets may be used directly in vivo without further modification, as they display an apparent resistance to nucleases. These GROs also require no special vehicle for delivery into cells, as nucleolin appears to specifically mediate transmembrane transport of GROs into cells (FIGS. 7A and B).

GROs may be of the form of DNA or RNA, as both polynucleotide forms are expected to form G-quartet structures in solution. Special considerations must be addressed for GROs comprising RNA as related to their efficient mode of delivery inside cells. Because RNA structure is exquisitely more sensitive to degradation by chemical and enzymatic processes than DNA, GROs comprising RNA may be optionally modified to render these molecules more stable to serum born degradative processes, like RNases. For example, modification of the 2′-hydroxyl group of RNA in GROs to created 2′-OMe modified GROs should render them resistant to nucleases and as effective NFκB inhibitors (except for SEQ ID NO:1). Alternatively, GROs comprised of RNA can be incorporated into genes that encode stable RNA products following their transcription, such as transfer RNA (tRNA). Such hybrid gene cassettes can serve as a novel intracellular therapeutic device following introduction of the gene cassette into cells using any one of a variety of gene or viral vector delivery approaches known to one of ordinary skill in the art. Expression of the GRO in the context of a stable RNA transcript, such as a tRNA, would result in accumulation of the GRO-containing transcript in the cytoplasm where it could exert its inhibitory effect upon the NFκB signaling pathway.

Chemical modification of GROs is also useful in the present invention. Those GRO aptamers that form less stable G-quartets or that display the propensity to form alternate conformations may require chemical modification of the polynucleotide structure to protect the oligonucleotide population from degradation by serum-borne nucleases. Such aptamers also may not be taken up by cells as readily as other GROs that form stable G-quartets, possibly owing to a reduced nucleolin-binding activity or reduced nucleolin-mediated transmembrane transport of these GROs. These GRO molecules would be expected to remain exposed to serum components outside the protective environment of cells, necessitating enhanced protective measures for their structures. Examples of chemical modifications that may impart greater stability to these molecules include terminal modifications of the sugar moieties (e.g., 5′- and 3′-amino groups) and phosphodiester modifications (e.g., phosphorothiolate groups). Optionally, GROs may be administered with additional components that protect the integrity of GRO structure (e.g., nuclease inhibitors) or that increase cellular uptake of GROs in particular treatment modalities (e.g., chloroquine).

Alternative modes of delivery of GROs into cells may be useful in the present invention. The structural integrity of the most robust GROs may not be amenable to certain therapy modes, such as treatment of inflammation of the GI tract. Furthermore, the distribution of nucleolin on the cell plasma membrane also may differ according to cell type, requiring alternative modes for intracellular delivery. In these contexts, the oligonucleotides may be encapsulated in suitable vehicles to further protect their structural integrity as well as to promote their delivery inside cells. Preferred vehicles include liposomes, lipid vesicles, microparticles, and the like.

Lipid vesicles resemble plasma membranes, and they can be made to fuse with cell membranes. Most liposomes and multilamellar vesicles are not readily fusogenic, mainly because the stored energy of the vesicle radius of curvature is minimal. Preferred lipid vesicles include small unilamellar vesicles. The small unilamellar vesicles contemplated for encapsulating GROs are very fusogenic, because they have a very tight radius of curvature. The average diameter of a small unilamellar vesicle is 5 nm to 500 nm; preferably 10 nm to 100 nm, more preferably 20 nm to 60 nm, including 40 nm. This size allows vesicles to pass through the gaps between endothelial cells, thereby permitting systemic delivery of GRO-containing vesicles following intravenous administration. Useful vesicles may vary greatly in size and are selected according to a specific application with a GRO.

Small unilamellar vesicles can be readily prepared in vitro using procedures available in the art (54, 55). The compositions from which the vesicles are formed contain a phospholipid which is a stable vesicle former, preferably together with another polar lipid, and optionally with one or more additional polar lipids and/or raft formers. Preferred phospholipids that are stable vesicle formers include 1-palmitoyl-2-docosahexaenoyl-sn-glycero-3-phosphocholine and 1,2-dioleoyl-sn-glycero-3-phosphocholine. Preferred polar lipids include: 1-palmitoyl-2-oleoyl-sn-glycero-3-phosphate, 1,2-dioleoyl-sn-glycero-3-ethylphosphocholine, 1,2-dioleoyl-sn-glycero-3-phosphoethanolamine, 1,2-dioleoyl-sn-glycero-3-[phospho-1-serine], a typical sphingomyelin, 1,2-dimyristoyl-sn-glycerol, and 1-palmitoyl-2-hydroxy-sn-glycero-3-phosphocholine. Other preferred polar lipids include phosphatidyl serine, phosphatidyl glycerol, mixed chain phosphatidyl choline, phosphatidyl ethanol, and phospholipids containing decosahexaenoic acids. One example of a preferred raft former is cholesterol.

Additional methods for preparing small unilamellar vesicles are described in U.S. application Ser. No. 10/397,048, A DIRECT CELLULAR ENERGY DELIVERY SYSTEM, to William D. Ehringer and Sufan Chien, filed Mar. 25, 2003. This application is hereby incorporated by reference in its entirety.

The GROs may be prepared as pharmaceutical compositions. Such compositions typically include GROs and a pharmaceutically acceptable carrier. A “pharmaceutically acceptable carrier” includes any and all solvents, dispersion media, coatings, antibacterial and antifungal agents, isotonic and absorption delaying agents, etc., compatible with pharmaceutical administration. Preferred examples of such carriers or diluents include, but are not limited to, water, saline, finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions. For example, inflammation is often associated with wounds, and becaplermin, as found in REGRANEX®, can be used to promote wound healing in conjunction with administration of GROs to reduce the inflammatory condition.

The efficacy of treating an inflammation with a GRO can be determined by measuring a change in expression of an NFκB-activated gene before and after administration of the GRO. An amount of a GRO effective for either acute or chronic inflammation therapy is an amount that reduces expression of an NFκB-activated gene, as assessed by measuring an NFκB-activated gene expression profile in a treated inflammatory lesion or condition. Optionally, an amount of a GRO effective for either acute or chronic inflammation therapy is an amount that reduces the extent of the inflammatory lesion or condition following administration of a pharmaceutical composition containing a GRO, as assessed upon direct examination.

The present invention contemplates administering a pharmaceutical composition containing a GRO in a dosage range of 1 mg of GRO per kg body weight to 5 mg of GRO per kg body weight. More preferably, the administration of a pharmaceutical composition containing a GRO in a dosage range of 1 mg, 1.5 mg, 2.0 mg, 2.5 mg, 3.0 mg, 4.0 mg and 5 mg of GRO per kg body are contemplated by the present invention. Further, the present invention contemplates administering a pharmaceutical composition containing a GRO periodically over 1, 2, 4, 6, 8, 10, 12, or 14 days for acute inflammation and preferably longer for chronic inflammation.

For pharmaceutical compositions that contain a GRO in combination with another anti-inflammatory agent, the preferred dosage levels of the anti-inflammatory agent will be limited to its effective dosage range when used independently of a GRO. Preferred dosage requirements of corticosteroids vary among individuals and diseases being treated. Preferably, the lowest possible effective dose is used. Preferably, an effective dose of a glucocorticoid is in the range of 0.005%-1.0% (topical cream) or 0.25 mg-500 mg (tablet), depending upon the method of administration, the glucocorticoid involved, and the disease treated. Dosage requirements of a typical nonsteroidal anti-inflammatory agent will also vary according to the type of drug and the inflammatory condition being treated. For aspirin, 500 mg-4,000 mg is the preferred range for recommended doses ingested daily. For ibuprofen, 200 mg-600 mg is the preferred range for recommended doses ingested daily. For naproxin, 125 mg-500 mg is the preferred range for recommended doses ingested daily. For nabumetone, 1 g-2 g is the preferred range for recommended doses ingested daily.

An effective dose of a flavonoid will vary depending upon the flavonoid and the inflammatory condition being treated. For example, a glass of red wine typically contains 650 μg of resveratol. Preferably, the adult daily dosage of resveratol is 2 to 2.5 milligrams. Optionally, tablet or capsule formulations of resveratol useful for the present invention have preferably 1 to 10 milligrams of the flavonoid.

For pharmaceutical compositions that contain a GRO in combination with an inhibitor of prostaglandin synthesis, such as an inhibitor of the COX-2 enzyme, the preferred dosage levels will mirror those found to be therapeutically effective when used independently of a GRO. For celecoxib, a capsule for oral administration contains preferably 1 mg to 400 mg of drug. More preferably, a celecoxib formulation may contain 100 mg, 200 mg, or 400 mg of drug. For rofecoxib, a formulation for daily oral administration contains preferably 1 mg to 50 mg of the drug. More preferably, a rofecoxib formulation may 12.5 mg, 25 mg, or 50 mg of the drug in tablet form, whereas a 5 mL suspension for daily oral administration may contain 12.5 mg or 25 mg of the drug. For valdecoxib, a tablet formulation may contain 1 mg to 20 mg of drug. More preferably, valdecoxib tablets prepared for once or twice daily oral administration may contain 10 mg or 20 mg of the drug.

A pharmaceutical composition is formulated to be compatible with the intended route of administration, including intravenous, intradermal, subcutaneous, oral, inhalation, transdermal, topical, transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

Injection provides a direct and facile route of administration, especially for tissue that is below the skin. Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, such as parabens, chlorobutanol, phenol, ascorbic acid, and thimerosal, can control microorganism contamination. Isotonic agents, such as sugars, polyalcohols such as manitol, sorbitol, and sodium chloride can be included in the composition.

Sterile injectable solutions or dispersions can be prepared by incorporating GROs in an appropriate solvent with one or a combination of ingredients, followed by sterilization. Sterile powders for the preparation of sterile injectable solutions may be prepared by vacuum drying and freeze-drying that yield a powder and any desired ingredient from sterile solutions.

Oral compositions generally include an inert diluent or an edible carrier. For the purpose of oral administration, the GROs can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

For administration by inhalation, the compounds are delivered as an aerosol spray from a nebulizer or a pressurized container that contains a suitable propellant, e.g., a gas such as carbon dioxide.

Systemic administration can also be mucosal or dermal. For mucosal or dermal administration, penetrants that can permeate the target barrier(s) are selected. Mucosal penetrants include, detergents, bile salts, and fusidic acid derivatives. Nasal sprays or suppositories can be used for mucosal administration. For dermal administration, the GROs are formulated into ointments, salves, gels, or creams. The GROs can also be prepared in the form of suppositories (e.g., with bases such as cocoa butter and other glycerides) for vaginal delivery or retention enemas for rectal delivery. Similarly, implantable drugs containing GROs may be used for dermal administration. A suitable delivery apparatus includes a patch, an implantable drug delivery device, a syringe, and a douche. Such devices permit localized administration at the site of the inflammatory lesion. For example, the delivery device may be a patch that contacts the patient's elbow and the drug is delivered locally to treat arthritis of the elbow Optionally, systemic administration is possible whereby the delivery device contacts the patient at a site remote from the site of the inflammatory lesion. For example, the delivery device may be an implantable drug delivery device on patient's arm and the drug is delivered systemically to treat an inflammatory condition of the intestine, such as Crohn's disease.

The nucleic acid molecules used in the invention, such as GROs comprise of RNA, can be inserted into vectors and used as gene therapy vectors. Gene therapy vectors can be delivered to a subject by, for example, intravenous injection, local administration (61), or by stereotactic injection (62). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Oral formulations or parenteral compositions in unit dosage form can be created to facilitate administration and dosage uniformity. Unit dosage form refers to physically discrete units suited as single doses for a subject, containing a effective quantity of GROs in association with a pharmaceutical carrier.

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit better long-term storage.

The reagents included in the kits can be supplied in containers of any sort such that the life of the different components are preserved and are not adsorbed or altered by the materials of the container. For example, sealed glass ampoules may contain buffer that has been packaged under a neutral non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include bottles that may be fabricated from similar substances, such as ampules, and envelopes that may consist of foil-lined interiors, such as aluminum or an alloy. Other containers include test tubes, vials, flasks, bottles, syringes, etc. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

Kits may also be supplied with instructional materials. Instructions may be printed on paper or other substrate, and/or may be supplied as an electronic-readable medium, such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

### EXAMPLES

The following examples are presented to aid the practitioner, although other methods, techniques, cells, reagents, and approaches can be used. These examples should not be construed to limit the invention in any manner.

### Example 1

### Activity and Specificity of GROs in Malignant Cells

All oligonucleotides (Oligos Etc.) contain phosphodiester backbones and 3′-C-3, or 3′-C-6 aminoalkyl modifications. The oligonucleotides were resuspended in water and sterilized by filtration through a 0.2 μm filter. Stock solutions of 400 or 500 μM were aliquoted and stored at −20° C. The integrity of the oligonucleotides was verified by 5′-radiolabeling followed by polyacrylamide gel electrophoresis.

The therapeutic potential of SEQ ID NO:10 for the treatment of hormone-refractory prostate cancer was tested in a DU145 xenograft model in nude mice. Male mice were inoculated subcutaneously with 10⁷ DU145 cells. After formation of small tumors (typically four days after inoculation), treatment was administered by intraperitoneal (i.p.) injection of SEQ ID NO:10, SEQ ID NO:18, or buffer lacking an oligonucleotide. Specifically, mice received either 20 μg or 100 μg of oligonucleotide (corresponding to approximately 1 mg oligonucleotide/kg body weight or 5 mg oligonucleotide per kg body weight, respectively) in 100 μl buffer i.p. on day 4, 5, 6, 8, 10, and 12 after receiving the tumor cells. Tumor volume was monitored by caliper measurement and mice were euthanized on day 14.

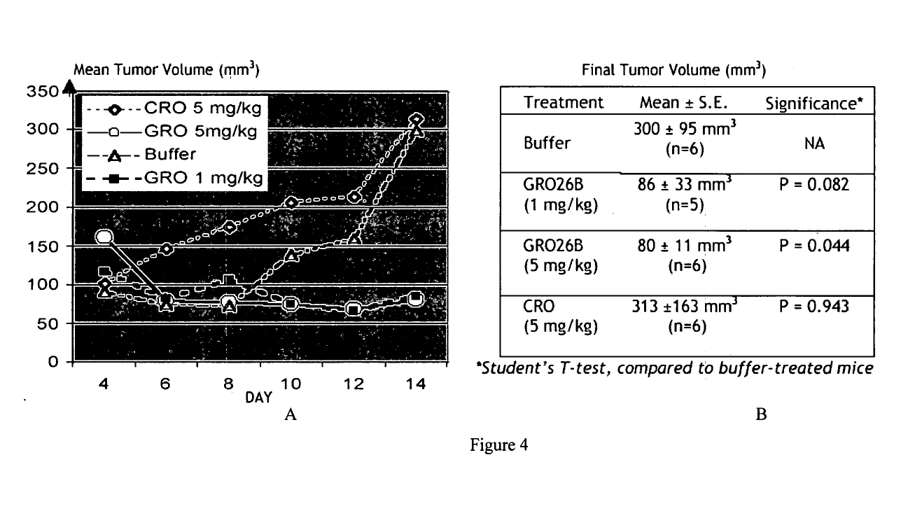

FIG. 4 demonstrates that buffers containing SEQ ID NO:10 effectively inhibited tumor growth and displayed a reduced final tumor volume, as compared to buffers containing either SEQ ID NO:18 or no oligonucleotide. This figure also shows the potent in vivo activity of SEQ ID NO:10 using oligonucleotide concentrations well below those typically required for tumor growth inhibition using antisense oligonucleotides. For example, an amount of antisense oligonucleotides corresponding to 25 mg oligonucleotide per kg body weight was required for effective treatment in the same DU145 model (56). Furthermore, the concentration range of GRO26B [SEQ ID NO:10] used in this experiment is well within the concentration range safe for humans (57). Finally, unmodified SEQ ID NO:10 was more effective than SEQ ID NO:10 that contained phosphorothiolate modifications.

### Example 2

### Identification of NEMO as a GRO-Associated Protein

Nuclear and S-100 extracts were prepared from HeLa cells according the protocol of Coqueret et al. (58). The nuclear and S-100 extracts (250 μg) were then incubated with 5′-biotinylated SEQ ID NO:1 or SEQ ID NO:8 for 30 minutes at 37° C. The GRO-protein complexes were then isolated using streptavidin-coated magnetic beads (Promega). The precipitated proteins were eluted from the beads by addition of SDS sample loading buffer and incubation at 65° C. for 15 minutes. Proteins were then separated on an 8% polyacrylamide SDS-PAGE and visualized by silver staining. Bands representing SEQ ID NO:1 specific binding proteins were then excised from the gel and prepared for mass spectrometric analysis.

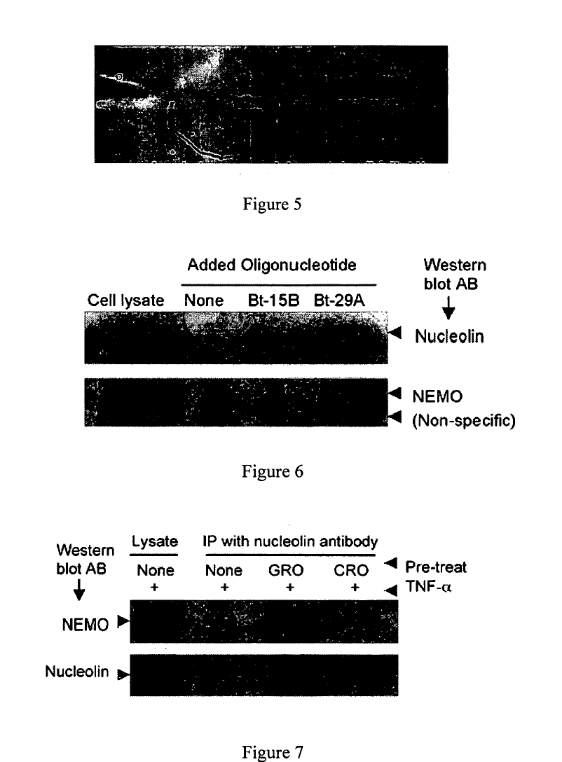

To obtain peptides for mass spectrometric analysis, silver stained bands were excised from gels and digested with trypsin according to a modified protocol from Jensen et al. (59). Briefly, the excised bands were incubated for 15 minutes in 100 nM NH₄HCO₃ and 50% acetonitrile and then dried at room temperature. Proteins were then reduced by incubation with 20 mM DTT at 56° C. for 45 minutes, followed by alkylation with 65 mM iodoacetamide in the dark for 30 minutes at room temperature. Bands were then incubated with for 15 minutes in 50 mM NH₄HCO₃ and 50% acetonitrile and dried at room temperature. The proteins were then hydrolyzed by incubation in 20 ng of modified trypsin (Promega) at 37° C. overnight. Trypsin-generated peptides were then applied by a thin-film spotting technique for MALDI-MS analysis using α-cyanohydroxycinnamic acid as a matrix on stainless steel targets as described by Jensen et al. (59). Mass spectral data were obtained with Tof-Spec 2E instrument (Micromass) and a 337-nm N₂ laser at 20 to 35% power in reflector mode. Peptide masses obtained were used to search the National Center for Biotechnology Information (NCBI) database to identify the proteins. FIG. 5 shows a typical example of this type of experiment and several GRO-binding proteins that includes NEMO, which could be reliably identified (p<0.05) by their MALDI-TOF mass spectrometry fingerprints.

To confirm NEMO as a GRO-associated protein, the following experiment was done. Hela cells were plated in Dulbecco's modified Eagle's medium (DMEM, Invitrogen) supplemented with 10% (v/v) fetal bovine serum (FBS, Invitrogen) and 1% penicillin/streptomycin solution (Invitrogen) and grown to 50% confluence in 25-mm culture flasks. The cells were treated with cell media containing 3 μM of 5′-biotinylated oligonucletoide (SEQ ID NO:10 or SEQ ID NO:18) for 2 hours at 37° C. The cells were then washed with phosphate buffered saline (PBS, Invitrogen) and lysed with lysis buffer (Promega). After 1 freeze-thaw cycle, the genomic DNA was sheared using a fine gauge needle. Streptavidin-coated magetic beads (Promega) were added to the cell lysate and incubated for 10 minutes at room temperature. Beads were captured and unbound sample was removed by repeated washing. The precipitated proteins were eluted from the beads by addition of SDS sample loading buffer and incubation at 65° C. for 15 minutes. The samples were then resolved on an 8% polyacrylamide SDS-PAGE and transferred to polyvinylidene diflouoride (PVDF) membrane.

PVDF membranes were blocked with 5% nonfat dried milk in PBST (0.1% Tween 20 in PBS) for 1 hour at room temperature. The membranes were then incubated with primary antibody (1:1000 anti-nucleolin (Santa Cruz) or 1:500 anti-NEMO (US Biological) in PBST) at room temperature for 1 hour. After sufficient washing, the membranes were then incubated with horseradish peroxidase-conjugated goat anti-mouse antibody for 45 minutes at room temperature, and the bands were detected using enhanced chemiluminescence (ECL, Amersham Biosciences).

### Example 3

### Identification of a GRO-Nucleolin-NEMO Ternary Complex

Hela cells were plated at a density of 2×10⁵ cells per well in a 6-well plate. After incubation of 18 hours to allow adherence, the cells were washed with PBS and treated with culture medium containing 10 μM oligonucleotide. The S-100 extracts from the treated cells were then extracted at the indicated time points according to the method of Coqueret et al. (58). One hundred micrograms of the S-100 extracts were incubated with 10 μg of anti-nucleolin antibody (Santa Cruz) for 2 hours at 4° C. Goat anti-mouse coated magnetic beads (Pierce) were then added to the samples and incubated for 1 hour at 4° C. The beads were captured and the unbound sample was removed by sufficient washing. The precipitated proteins were eluted from the beads by the addition of SDS sample loading buffer and heating at 65° C. for 15 minutes. The samples were then run on a 8% polyacrylamide SDS-PAGE and transferred to a PVDF membrane. The membrane was then probed for the presence of NEMO and nucleolin as described in Example 2.

FIG. 7 illustrates that NEMO is selectively immunoprecipitated with nucleolin in those cells pre-treated with SEQ ID NO:10, but not in cells pre-treated with SEQ ID NO:18 or without any oligonucleotide. This experiment demonstrates that a nucleolin-NEMO complex exists in cells treated with SEQ ID NO:10, presumably as a ternary complex involving SEQ ID NO:10-nucleolin-NEMO.

### Example 4

### GROs Block NFκB Signaling

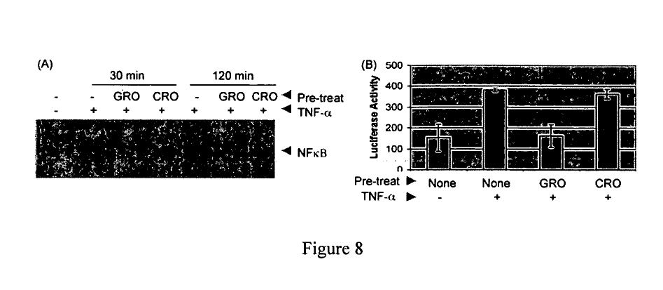

The following example demonstrates that GROs are capable of blocking NFκB signaling through inhibiting NFκB activation. Hela cells were plated at a density of 2×10⁵ cells per well in a 6-well plate. After incubation of 18 hours to allow adherence, the cells were washed with PBS and treated with culture medium containing 10 μM oligonucleotide. Following incubation of 1 hour, recombinant human tumor necrosis factor alpha (TNF-α) (R & D Systems, Inc.) was added directly to the culture medium at a final concentration of 7.5 ng/ml. Nuclear extracts from the treated cells were then extracted at the indicated time points according to the method of Coqueret et al. (1). Single-stranded oligonucleotides containing the NFκB upstream response element (URE) (5′-TGCAGGAGGTCCGGCTTTTCCCCAACCCCCC-3′) [SEQ ID NO:40] and its antisense strand [SEQ ID NO:41] (Integrated DNA Technologies, Inc.) were annealed by boiling for 3 minutes and cooling slowly to room temperature. The double stranded NF-κB URE was then end-labeled using T4 kinase (Invitrogen) and [γ-³²P]-dATP. Nuclear extracts (5 μg) were incubated with labeled probe and binding buffer (20 mM HEPES, pH 7.9, 50 mM KCl, 1 mM EDTA, 5% glycerol, 5 mM DTT, and 1 mM PMSF) for 45 minutes at room temperature. DNA-protein complexes were analyzed on a 5% native polyacrylamide gel in TBE buffer (90 mM TRIS borate, 2 mM EDTA). The gel was subsequently dried and subjected to autoradiography. FIG. 8A illustrates the results from a typical experiment. GRO more effectively inhibited NFκB-DNA complex formation than did a control oligonucleotide.

To confirm that the in vitro electrophoretic mobility shift assay accurately portrayed the inability of NFκB to bind productively to cognate sites in vivo in cells treated with GRO, the activity of a NFκB-driven luciferase reporter gene was evaluated (60). HeLa cells were plated in 24-well plates at a density of 2×10⁴ per well and were transiently transfected by superfect reagent (Qiagen) with a NFκB luciferase reporter plasmid or a control null luciferase plasmid (a kind gift of Dr. Sham Kakar). Superfect reagent (5 μl/1 g of plasmid DNA) and the plasmid DNA (1 μg) were added to the wells in antibiotic-free and serum-free DMEM. After 3 hours of incubation, the cells were washed with PBS, followed by the addition of culture medium supplemented with the appropriate serum and antibiotics. Twenty-four hours after the transfection, olionucleotides of SEQ ID NO:10 and SEQ ID NO:18 were added directly to the medium of parallel cultures at a final concentration of 10 μM and the cultures were allowed to incubate for 1 hour. Recombinant TNF-α (R & D Systems, Inc.) was then added to the medium and incubation continued for 6 hours. Cells were harvested in reporter lysis buffer (Promega Corp.). The cell lysate was added to luciferase reagent (20 mM Tricine, 1.07 mM MgCO₃, 2.67 mM MgSO₄, 0.1 mM EDTA, 33.3 mM DTT, 530 μM ATP, 270 μM Coenzyme A, and 470 μM Luciferin), and the luciferase activity was then measured by luminometer. FIG. 8B illustrates that SEQ ID NO:10 inhibited TNFα-mediated activation of the NFκB-responsive reporter gene, whereas SEQ ID NO:18 was ineffective under comparable conditions.

### REFERENCES

1. Makarov S S. “NF-κB as a therapeutic target in chronic inflammation: recent advances.” Mol Med Today 6:441-8 (2000).
2. Pahl H L. “Activators and target genes of Rel/NF-kB transcription factors.” Oncogene 18:6953-66 (1999).
3. Baldwin A S, Jr. “The NF-κB and IκB proteins: new discoveries and insights.” Annu. Rev. Immunol. 14:649-83 (1996).
4. Ghosh S, May M J, Kopp E B. “NF-κB and Rel proteins: evolutionarily conserved mediators of immune responses.” Annu. Rev. Immunol. 16:225-60 (1998).
5. Whiteside S T, Israel A. “IκB proteins: structure, function and regulation.” Semin. Cancer Biol. 8:75-82 (1997).
6. Karin M. Ben-Neriah Y. “Phosphorylation meets ubiquitination: the control of NF-κB activity.” Annu. Rev. Immunol. 18:621-663 (2000).
7. Israel A. “The IKK complex: an integrator of all signals that activate NF-κB.” Trends Cell Biol. 10:129-33 (2000).
8. Rothwarf D M, Zandi E, Natoli G, Karin M. “IKK-γ is an essential regulatory subunit of the IκB kinase complex.” Nature 395:297-300 (1998).
9. Yamaoka S, Courtois G, Bessia C, Whiteside S T, Weil R, Agou F, Kirk H E, Kay R J, Israel A. “Complementation cloning of NEMO, a component of the IκB kinase complex essential for NFκB activation.” Cell 93:1231-40 (1998).
10. Rayet B, Gelinas C. “Aberrant rel/nfkb genes and activity in human cancer.” Oncogene 18:6938-47 (1999).
11. Mayo M W, Badwin A S. “The transcription factor NF-κB: control of oncogenesis and cancer therapy resistance.” Biochim. Biophys. Acta 1470:M55-M62 (2000).
12. Romashkova J A, Makarov S S. “NF-κB is a target of AKT in anti-apoptotic PDGF signalling.” Nature 401:86-90 (1999).
13. Hinz M, Krappmann D, Eichten A, Heder A, Scheidereit C, Strauss M. “NF-κB function in growth control: regulation of cyclin D1 expression and G₀/G₁-to-S-phase transition.” Mol. Cell. Biol. 19:2690-8 (1999).
14. Barkett M, Gilmore T D. “Control of apoptosis by Rel/NF-κB transcription factors.” Oncogene 18:6910-24 (1999).
15. Gasparian A V, Yao Y J, Lu J, Yemelyanov A Y, Lyakh L A, Slaga T J, Budunova I V. “Selenium compounds inhibit IκB kinase (IKK) and nuclear factor-κB (NF-κB) in prostate cancer cells.” Mol Cancer Ther. 1:1079-87 (2002).
16. Yamamoto Y, Gaynor R B. “Therapeutic potential of inhibition of the NF-κB pathway in the treatment of inflammation and cancer.” J. Clin. Invest. 107135-42 (2001).
17. Shen W, Waldschmidt M, Zhao X, Ratliff T, Krieg A M. “Antitumor mechanisms of oligodeoxynucleotides with CpG and polyG motifs in murine prostate cancer cells: decrease of NfkappaB and AP-1 binding activities and induction of apoptosis.” Antisense Nucleic Acid Drug Dev. 12:155-64 (2002).
18. Karin M. “New twists in gene regulation by glucocorticoid receptor: is DNA binding dispensible?” Cell 93:487-90 (1998).
19. Yang J P, Merin J P, Nakano T, Kato T, Kitade Y, Okamoto T. “Inhibition of the DNA-binding activity of NF-κB by gold compounds in vitro.” FEBS Lett. 361:89-96 (1995).
20. Yin M J, Yamamoto Y, Gaynor R B. “The anti-inflammatory agents aspirin and salicylate inhibit the activity of IκB kinase-β.” Nature 396:77-80 (1998).
21. Epinat J C, Gilmore T D. “Diverse agents act at multiple levels to inhibit the Rel/NF-κB signal transduction pathway.” Oncogene 18:6896-6909 (1999).
22. Jobin C, Bradham C A, Russo M P, Juma B, Narula A S, Brenner D A, Sartor R B. “Curcumin blocks cytokine-mediated NF-κB activation and proinflammatory gene expression by inhibiting inhibitory factor I-κB kinase activity.” J. Immunol. 163:3474-83 (1999).
23. Tomita T, Takeuchi E, Tomita N, Morishita R, Kaneko M, Yamamoto K, Nakase T, Seki H, Kato K, Kaneda Y, Ochi T. “Suppressed severity of collagen-induced arthritis by in vivo transfection of nuclear factor kappa B decoy oligodeoxynucleotides as a gene therapy.” Arthritis Rheum. 42:2532-42 (1999).
24. Bantel H, Schmitz M L, Raible A, Gregor M, Schulze-Osthoff K. “Critical role of NF-κB and stress-activated protein kinases in steroid unresponsiveness.” FASEB J. 16:1832-34 (2002).
25. Kaltschmidt B, Uherek M, Wellmann H, Volk B, Katschmidt C. “Inhibition of NF-κB potentiates amyloid beta-mediated neuronal apoptosis.” Proc. Natl. Acad. Sci., U.S.A. 96:9409-14 (1999).
26. Bates P J, Kahlon J B, Thomas S D, Trent J O, Miller D M “Antiproliferative activity of G-rich oligonucleotides correlates with protein binding.” J. Biol. Chem. 274:26369-77 (1999).
27. Derenzini M, Sirri V, Trere D, Ochs R L. “The quantity of nucleolar proteins nucleolin and protein B23 is related to cell doubling time in human cancer cells.” Lab. Invest. 73:497-502 (1995).
28. Roussel P, Sirri V, Hernandez-Verdun D. “Quantification of Ag-NOR proteins using Ag-NOR staining on western blots.” J. Histochem Cytochem. 42:1513-7 (1994).
29. Pich A, Chiusa L, Margaria E. “Prognostic relevance of AgNORs in tumor pathology.” Micron. 31:133-41 (2000).
30. Trere D, Derenzini M, Sirri V, Montanaro L, Grigioni W, Faa G, Columbano G M, Columbano A. “Qualitative and quantitative analysis of AgNOR proteins in chemically induced rat liver carcinogenesis.” Hepatology 24:1269-73 (1996).
31. Data derived from genome.ucsc.edu (to locate nucleolin sequence at 2q37.1) and the Mitelman Database of Chromosome Aberrations in Cancer (cgap.nci.nih.gov/Chromosomes).
32. Srivastava M, Pollard H B. “Molecular dissection of nucleolin's role in growth and cell proliferation: new insights.” FASEB J. 13:1911-22 (1999).
33. Tuteja R, Tuteja N. “Nucleolin: a multifunctional major nucleolar phosphoprotein.” Crit. Rev. Biochem. Mol. Biol. 33:407-36 (1998).
34. Ginisty H, Sicard H, Roger B, Bouvet P. “Structure and functions of nucleolin.” J. Cell. Sci. 112 (Pt 6):761-72 (1999).
35. Yang C, Maiguel D A, Carrier F. “Identification of nucleolin and nucleophosmin as genotoxic stressresponsive RNA-binding proteins.” Nucleic Acids Res. 30:2251-60 (2002).
36. Daniely Y, Borowiec J A. “Formation of a complex between nucleolin and replication protein A after cell stress prevents initiation of DNA replication.” J. Cell Biol. 149:799-810 (2000).
37. Wang Y, Guan J, Wang H, Wang Y, Leeper D, Iliakis G. “Regulation of dna replication after heat shock by replication protein a-nucleolin interactions.” J. Biol. Chem. 276:20579-88 (2001).
38. Bates P J, Miller D M, Trent J O, Xu, X. “Method for the diagnosis and prognosis of malignant diseases,” U.S. patent application Ser. No. 10/118,854, filed in the USPTO on Apr. 8, 2002.
39. Barel M, Le Romancer M, Frade R. “Activation of the EBV/C3d receptor (CR2, CD21) on human B lymphocyte surface triggers tyrosine phosphorylation of the 95-kDa nucleolin and its interaction with phosphatidylinositol 3 kinase.” J. Immunol. 166:3167-73 (2001).
40. Larrucea S, Cambronero R, Gonzalez-Rubio C, Fraile B, Gamallo C, Fontan G, Lopez-Trascasa M. “Internalization of factor J and cellular signalization after factor J-cell interaction.” Biochem. Biophys. Res. Commun. 266:51-7 (1999).
41. Dumler I, Stepanova V, Jerke U, Mayboroda O A, Vogel F, Bouvet P, Tkachuk V, Haller H, Gulba D C. “Urokinase-induced mitogenesis is mediated by casein kinase 2 and nucleolin.” Curr. Biol. 9:1468-76 (1999).
42. Hovanessian A G, Puvion-Dutilleul F, Nisole S, Svab J, Perret E, Deng J S, Krust B. “The cell-surface expressed nucleolin is associated with the actin cytoskeleton.” Exp. Cell Res. 261:312-28 (2000).
43. Callebaut C, Blanco J, Benkirane N, Krust B, Jacotot E, Guichard G, Seddiki N, Svab J, Dam E, Muller S, Briand J P, Hovanessian A G. “Identification of V3 loop-binding proteins as potential receptors implicated in the binding of HIV particles to CD4(+) cells.” J. Biol. Chem. 273:21988-97 (1998).
44. Borer R A, Lehner C F, Eppenberger H M, Nigg E A. “Major nucleolar proteins shuttle between nucleus and cytoplasm.” Cell 56:379-90 (1989).
45. Martin P, Duran A, Minguet S, Gaspar M L, Diaz-Meco M T, Rennert P, Leitges M, Moscat J. “Role of zeta PKC in B-cell signaling and function.” EMBO J. 21:4049-57 (2002).
46. Zhou G, Seibenhener M L, Wooten M W. Nucleolin is a protein kinase C-zeta substrate. Connection between cell surface signaling and nucleus in PC12 cells.” J. Biol. Chem. 272:31130-7 (1997).
47. Turutin D V, Kubareva E A, Pushkareva M A, Ullrich V, Sud'ina G F. “Activation of NF-kappa B transcription factor in human neutrophils by sulphatides and L-selectin cross-linking.” FEBS Lett. 536:241-5 (2003).
48. Harms G, Kraft R, Grelle G, Volz B, Dernedde J, Tauber R. “Identification of nucleolin as a new L selectin ligand.” Biochem. J. 360(Pt 3):531-8 (2001).
49. Salazar R, Brandt R, Krantz S. “Binding of Amadori glucose-modified albumin by the monocytic cell line MonoMac 6 activates protein kinase C ε protein tyrosine kinases and the transcription factors AP-1 and NF-κB.” Glycoconj J. 18:769-77 (2001).
50. Sugano N, Chen W, Roberts M L, Cooper N R. “Epstein-Barr virus binding to CD21 activates the initial viral promoter via NF-κB induction.” J. Exp Med. 186:731-7 (1997).
51. Gil D, Gutierrez D, Alarcon B. “Intracellular redistribution of nucleolin upon interaction with the CD3ε chain of the T cell receptor complex.” J. Biol. Chem. 276:11174-9 (2001).
52. Weil R, Schwamborn K, Alcover A, Bessia C, Di Bartolo V, Israel A. “Induction of the NF-κB cascade by recruitment of the scaffold molecule NEMO to the T cell receptor.” Immunity 18:13-26 (2003).
53. Miranda G A, Chokler I, Aguilera R J. “The murine nucleolin protein is an inducible DNA and ATP binding protein which is readily detected in nuclear extracts of lipopolysaccharide-treated splenocytes.” Exp. Cell Res. 217:294-308 (1995).
54. Hauser H, Gains N. Spontaneous vesiculation of phospholipids: a simple and quick method of forming unilamellar vesicles. Proc. Natl. Acad. Sci., U.S.A. 79:1683-7 (1982).
55. Pitcher W H III, Huestis W H. “Preparation and analysis of small unilamellar phospholipid vesicles of a uniform size.” Biochem. Biophys. Res. Commun. 296:1352-5 (2002).
56. Wang H, Yu D, Agrawal S, Zhang R. “Experimental therapy of human prostate cancer by inhibiting MDM2 expression with novel mixed-backbone antisense oligonucleotides: In vitro and in vivo activities and mechanisms.” Prostate 54:194-205 (2003).
57. Chi K N, Gleave M E, Klasa R, Murray N, Bryce C, Lopes de Menezes D E, D'Aloisio S, Tolcher A W. “A phase I dose-finding study of combined treatment with an antisense Bcl-2 oligonucleotide (Genasense) and mitoxantrone in patients with metastatic hormone-refractory prostate cancer.” Clin. Cancer Res. 7, 3920-3927 (2001).
58. Coqueret O, Gascan H. “Functional interaction of STAT3 transcription factor with the cell cycle inhibitor p21WAF1/CIP1/SDI1.” J. Biol. Chem. 275:18794-800 (2000).
59. Jensen O, Wilm M, Shevchenko A, Mann M. “Peptide sequencing of 2-DE gel-isolated proteins by nanoelectrospray tandem mass spectrometry.” Methods Mol. Biol. 112:513-30 (1999).
60. Kikuchi E, Horiguchi Y, Nakashima J, Kuroda K, Oya M, Ohigashi T, Takahashi N, Shima Y, Umezawa K, Murai M. “Suppression of hormone-refractory prostate cancer by a novel nuclear factor KB inhibitor in nude mice.” Cancer Res. 63:107-10 (2003).
61. Nabel, E G, Nabel, G J. “Treatment of diseases by site-specific instillation of cells or site-specific transformation of cells and kits therefor.” U.S. Pat. No. 5,328,470. (1994).
62. Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.

1. A method of treating inflammation in a patient, comprising administering to the patient a composition comprising a GRO.

2. The method of claim 1, wherein the GRO has a nucleotide sequence comprising at least one member selected from the group consisting of SEQ ID NOs: 1, 10, 11, 26, 27, 28, 29, 30, 31, and 32.

3. The method of claim 1, wherein the patient is a mammal.

4. The method of claim 3, wherein the mammal is selected from the group consisting of a human, a dog, a cat, a cow, a sheep, a goat, a horse, a buffalo, and a pig.

5. The method of claim 1, wherein the inflammation is associated with an acute inflammatory condition.

6. The method of claim 5, wherein the acute inflammatory condition is selected from the group consisting of primary dysmenorrhea, acute alcoholic liver disease, and acute pancreatitis.

7. The method of claim 1, wherein the inflammation of Alzheimer's disease.

8. The method of claim 1, wherein the inflammation is associated with a chronic inflammatory disease.

9. The method of claim 8, wherein the chronic inflammatory disease is one member selected from the group consisting of rheumatoid arthritis, asthma, gastrointestinal tract disease, psoriasis, and atherosclerosis.

10. The method of claim 8, wherein the chronic inflammatory disease is at least one member selected from the group consisting of Crohn's disease, ulcerative colitis alcohol, chronic alcoholic liver disease, non-alcoholic steatohepatitis, and chronic pancreatitis.

11. The method of claim 1, wherein the composition further comprises an anti-inflammatory agent.

12. The method of claim 11 wherein the anti-inflammatory agent comprises at least one member selected from the group consisting of a corticosteroid, a nonsteroidal anti-inflammatory agent, a flavonoid, vitamin A, vitamin C, a cyclopentenone prostaglandin, tacrolimus, and cyclosporin A.

13. The method of claim 11, wherein the anti-inflammatory agent comprises a nonsteroidal anti-inflammatory agent.

14. The method of claim 13, wherein the nonsteroidal anti-inflammatory agent comprises at least one member selected from the group consisting of aspirin, ibuprofen, naproxen, and nabumetone.

15. The method of claim 13, wherein the nonsteroidal anti-inflammatory agent comprises a prostaglandin synthesis inhibitor.

16. The method of claim 15, wherein the prostaglandin synthesis inhibitor is a COX-2 inhibitor.

17-21. (canceled)

22. A pharmaceutical composition comprising:
an amount of a GRO effective for inflammation therapy;
an anti-inflammatory agent; and
a pharmaceutically acceptable carrier.

23-37. (canceled)

38. A method for providing chronic inflammation therapy to a mammal, comprising administering an effective amount of a pharmaceutical composition, comprising:
a vesicle, wherein the vesicle comprises an amount of a GRO effective for inflammation therapy;
an anti-inflammatory agent; and
a pharmaceutically acceptable carrier.

39-41. (canceled)

42. A method for determining the efficacy of treating inflammation with a GRO, comprising:
administering the GRO; and
measuring a change in an NFκB activity before and after administration of the GRO.

43-54. (canceled)

55. A pharmaceutical composition, comprising:
an amount of a GRO effective for inflammation therapy; and
a pharmaceutically acceptable carrier,
wherein the pharmaceutical composition is supplied as one selected from the group consisting of a suppository, a cream, an enema, and an aerosol.

56-61. (canceled)
